(19) United States
(12) Patent Application Publication
(10) Pub. No.: US 2019/0038856 A1
(43) Pub. Date: Feb. 7, 2019

(54) Medical cannabis and cannabinoids delivery device

(71) Applicant: Marvin Manuel Boone, JR., Orange Park, FL (US)

(72) Inventor: Marvin Manuel Boone, JR., Orange Park, FL (US)

(21) Appl. No.: 15/731,814

(22) Filed: Aug. 7, 2017

(51) Int. Cl. A61M 15/06; A24F 47/00; A61B 5/1172; A61M 15/00; A61K 36/185; A61K 9/00

(52) U.S. Cl. CPC A61M 15/06; A24F 47/004; A61B 5/1172; A61M 15/009; A61K 36/185; A61M 2205/127; A61M 2205/502; A61M 2205/609; A61M 2205/52; A61M 2205/583; A61K 9/0073

(57) ABSTRACT

Since some of the Individual States of the Federal Government no longer prosecute individuals for the possession or sale of medical marijuana, as long as compliance within the states sale regulations are followed, a new industry has emerged, Medical Cannabis.

Medical Cannabis is cannabis and cannabinoids that are prescribed by a medical doctor to their patents to alleviate certain types of nausea and vomiting associated with chemotherapy, also to improve appetite in HIV/AIDS patients and to reduce chronic pain and muscle spasms. This has produced many problems specifically dosage and dosage control. A cigarette from tip to filter contains about one mg nicotine and is controlled by the cigarette industry, as this is the typical amount to addict a person. Because there are not measures to control the amount of medical cannabis and cannabinoids delivered by use of a Vaporizer, Atomizer, Cartomizer, or Clearomizer I have invented the MEDICAL CANNABIS or CANNABINOIDS DELIVERY DEVICE (MCDD) which delivers a doctor's prescribed dosage and at the prescribed time to only the authorized user.

### CROSS REFERENCE TO RELATED APPLICATIONS

This application claims the benefit of U.S. Provisional Application No. 62/377,550, filed 20 Aug. 2016, the entire disclosures of which are incorporated herein by reference.

### STATEMENT REGARDING FEDERALLY SPONSORED RESEARCH OR DEVELOPMENT

Not Applicable.

### REFERENCE TO A MICROFICHE APPENDIX

Not Applicable.

### BACKGROUND OF THE INVENTION

This invention relates generally to a Medical Cannabis Delivery Device such as but not limited to inhaling and exhaling the vapor produced by an electric device referred to such as but not limited to a Vaporizer, Atomizer, Cartomizer, or a Clearomizer. If one vaporizes a piece of medical cannabis wax it is called an atomizer; or if an oil saturated polyfilled material is wrapped around a heating source then it's a cartomizer; or if oil is dripped from a separate holding tank using a silica wick then it's a clearomizer.

Medical Cannabis is cannabis and cannabinoids prescribed by a medical doctor to their patents for such as but not limited to alleviation of certain types of nausea and vomiting associated with chemotherapy, also to improve appetite in HIV/AIDS patients and to reduce chronic pain and muscle spasms.

Said invention is further more and specifically related to the U.S. Patent classification definitions technical field of but not limited to; Class D24, MEDICAL AND LABORATORY EQUIPMENT/Subclass 100, PHARMACEUTICAL PRODUCT (DESIGNED FOR MEDICINAL DRUGS); and Class D24, MEDICAL AND LABORATORY EQUIPMENT/Subclass 107 EQUIPMENT FOR DIAGNOSIS, ANALYSIS OR TREATMENT; and Class D24 MEDICAL AND LABORATORY EQUIPMENT/Subclass 110 VAPORIZER.

### PRIOR ART

Viewing prior art of U.S. Pat. No. 586,149 dated Jul. 13, 1897 and Titled: HYGIENIC PIPE discloses an apparatus in FIG. 1. Illustrating a smoking device that is adequately described in said '149 specifications.

Citing '149 beginning Column 1, Line 10, “ . . . improvements on hygienic pipes . . . ” and Line 18, “ . . . to construct a pipe which can be smoked while lying down and one by which the smoke can be purified and cooled before being inhaled . . . ”, and line 68 “ . . . placed upon a suitable stand “M” which supports a heating lamp N . . . ”.

Said '149 neither discuss the mechanics of smoke to air ratio nor how a patent could use '149 to deliver a reliably measurable dose of medicine.

Another example of prior art is from 1971, U.S. Pat. No. 3,587,573 Titled: INHALATION DEVICE WITH RUPTURABLE MICROSCOPIC CAPSULES by John Gene Flack.

Said '573 ABSTRACT describes an alternative way for an inhalation device to be used; however, it too lacks a method for controlling the release of the contents. Viewing of Column 1, Line 43 “ . . . microcapsules 14 . . . ” Medicine contained within Column 2, Claim 1 “ . . . a plurality of microscopic pressure rupturable capsules . . . ” are uncontrollably released when ruptured as well as a percentage of said microcapsules 14 neither would be rendered completely ruptured and some said microcapsules 14 would not be ruptured at all.

While both '149 and '573 claim the benefit of new and unique inventions, and while they have dramatically changed cigarette smoking from the traditional filter and paper wrapped tobacco, which is ignited and then inhaled and exhaled, they have not done so without creating some problems which are now inherent in virtually all electronic vaporizing device.

Furthermore; said '149 and '573 as well as other devices are not securable allowing unauthorized use of non-prescribe medicine. Said prior art does not have a means to record such as but not limited to usage, potency or draw strength. Said prior art neither identify the patient nor display a patient's prescription. Said prior art does not have display screens illustrating the aforementioned. Said prior art does not contain computer communication ports, and neither the medical doctor nor the pharmacist can adjust the strength, the dosage or the duration between said doses. To date neither Vaporizer, Atomizer, Cartomizer, nor a Clearomizer can deliver a reliably measurable dose of medical cannabis or cannabinoids medicine. Additionally, '149 and '573 as patented and as well as many other patents would not be able to crossover to medical use.

Accordingly, a need remains for MEDICAL CANNABIS AND CANNABINOIDS DELIVERY DEVICE in order to overcome the above-noted shortcomings. The non-limiting description satisfies such a need by providing an apparatus that is convenient and easy to use, lightweight yet durable in design, versatile in its applications, and designed to deliver a reliably measurable dose of medical cannabis and cannabinoids.

### BRIEF SUMMARY OF THE INVENTION

In view of the foregoing background, it is therefore an object of the non-limiting exemplary embodiments to provide a brief summary of the invention; some of the advantages are construction, security and the ability to record such as but not limited to usage, potency and draw length, the identity of patient and said patient's prescription on a display screen. Also advantages is only said medical doctor or pharmacist can adjust the strength, said dosage or said duration between said doses as well as view recorded patient data by means of a computer communication port. Additionally, MEDICAL CANNABIS AND CANNABINOIDS DELIVERY DEVICE delivers a reliably measurable dose of medicine. Embodiments described below demonstrate how said MEDICAL CANNABIS AND CANNABINOIDS DELIVERY DEVICE solves many of said prior art problems.

Described in one embodiment is said MEDICAL CANNABIS AND CANNABINOIDS DELIVERY DEVICE construction.

An additional embodiment of the invention discloses how MEDICAL CANNABIS AND CANNABINOIDS DELIVERY DEVICE reduces unauthorized use.

Yet in another embodiment is describe how MEDICAL CANNABIS AND CANNABINOIDS DELIVERY DEVICE records such as but not limited to usage, potency and draw length; and can identify said patient and said patient's prescription on a display screen.

An additional embodiment of the invention describes only said medical doctor or pharmacist can adjust the strength, said dosage or said duration between said doses as well as view recorded patient data by means of a computer communication port.

In addition, an embodiment disclosing how said MEDICAL CANNABIS AND CANNABINOIDS DELIVERY DEVICE delivers a reliably measurable dose of medicine.

These and other objects, features, and advantages of the invention are provided by description of the preferred embodiments.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

It is noted the purpose of the foregoing abstract is to enable the U.S. Patent and Trademark Office and the public generally, especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

### BRIEF DESCRIPTION OF THE VIEW OF THE DRAWING

The novel features believed to be characteristic of this invention are set forth with particularity in the appended claims. The invention itself, however, both as to its organization and method of operation, together with further objects and advantages thereof, may best be understood by reference to the following description taken in connection with the accompanying drawings in which:

FIG. 1 is an isometric view of said MEDICAL CANNABIS AND CANNABINOIDS DELIVERY DEVICE in accordance with the non-limiting exemplary embodiments.

Those skilled in the art will appreciate that the figures are not intended to be drawn to any particular scale; nor are the FIGURES intended to illustrate every embodiment of the invention. The invention is not limited to the exemplary embodiments depicted in the FIGURES or the shapes, relative sizes or proportions shown in the FIGURES.

### DETAILED DESCRIPTION OF THE INVENTION

The non-limiting exemplary embodiments will now be described more fully hereinafter with reference to the accompanying drawings, in which a preferred embodiment of the invention is shown. This invention may, however, be embodied in many different forms and should not be construed as limited to the embodiment set forth herein. Rather, these embodiments are provided so that this application will be thorough and complete, and will fully convey the true scope of the invention to those skilled in the art. Like numbers refer to like elements throughout the FIGURES.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “non-limiting exemplary embodiments” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The below disclosed subject matter is to be considered illustrative, and not restrictive, and the appended claims are intended to cover all such modifications, enhancements, and other embodiments which fall within the true scope of the non-limiting exemplary embodiments. Thus, to the maximum extent allowed by law, the scope of the non-limiting exemplary embodiments is to be determined by the broadest permissible interpretation of the following claims and their equivalents, and shall not be restricted or limited by the foregoing detailed description.

References in the specification to “one embodiment”, “an embodiment”, “a preferred embodiment”, “an alternative embodiment” and similar phrases mean that a particular feature, structure, or characteristic described in connection with the embodiment is included in at least an embodiment of the invention. The appearances of the phrase “non-limiting exemplary embodiment” in various places in the specification are not necessarily all meant to refer to the same embodiment.

Directional and/or relationary terms such as, but not limited to, left, right, nadir, apex, top, bottom, vertical, horizontal, back, front and lateral are relative to each other and are dependent on the specific orientation of an applicable element or article, and are used accordingly to aid in the description of the various embodiments and are not necessarily intended to be construed as limiting.

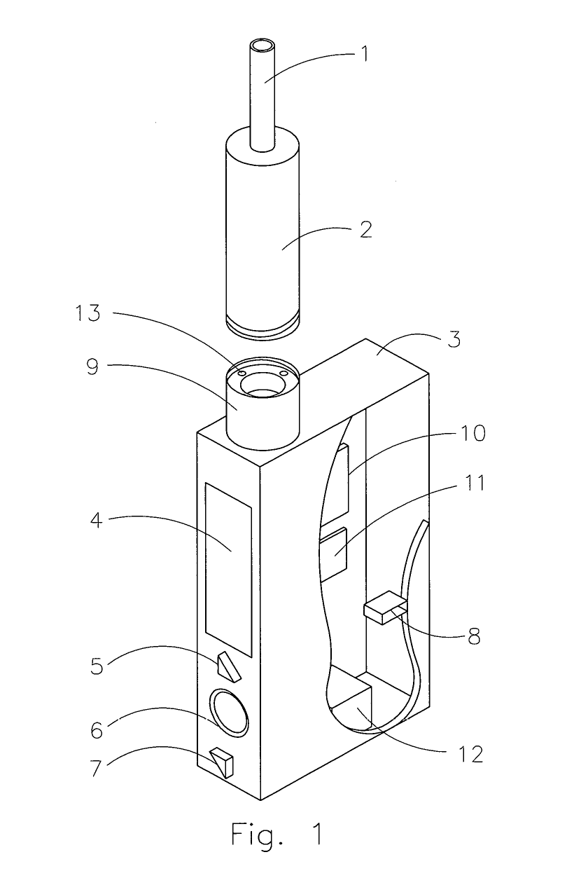

The apparatus of this invention is referred to generally in FIG. 1 intends to provide a isometric view showing MEDICAL CANNABIS AND CANNABINOIDS DELIVERY DEVICE. It should be understood that the non-limiting exemplary embodiments may be used to describe similar apparatus and many different types of devices and should not be limited to the uses described herein. The descriptor nomenclature legend that follows is not meant to be all encompassing but to aid the reader in understanding the FIGURES within the drawings.
1. Nozzle; and
2. Vaporizer, Atomizer, Cartomizer, or a Clearomizer Chamber; and
3. Tamper Resistant Exterior Case; and
4. Digital Display; and
5. Forward/Up Communication Button; and
6. Fingertip Biometric Scanner; and
7. Reverse/Down Communication Button; and
8. Charging and Communication Port; and
9. Heating Element Receptacle; and
10. Onboard Computer Processor; and
11. Evaluation Computer Processor; and
12. Power Source; and
13. Pass By Vapor Sensing Port(s).

Viewing FIG. 1, Reader can observe said Tamper Resistant Exterior Case 3 encasing and confining at least an Onboard Computer Processor (OCP) 10, and at least an Evaluation Computer Processor (ECP) 11, and a Power source 12; and said Tamper Resistant Exterior Case 3 having at least an external Digital Display 4, and Forward/Up Communication Button 5, and Fingertip Biometric Scanner 6, and Reverse/Down Communication Button 7; and Charging and Communication Port 8, and Heating Element Receptacle 9; and Vaporizer, Atomizer, Cartomizer, or Clearomizer Chamber 2; and Nozzle 1. Said Heating Element Receptacle 9 contains at least one vapor sensing port 13.

Viewing FIG. 1 of said invention MEDICAL CANNABIS AND CANNABINOIDS DELIVERY DEVICE's Tamper Resistant Exterior Case 3 is constructed from such as but not limited to a dense, vibration absorbing material, of structurally complementary substances, especially a chemical composition with physical and thermal properties, combined to produce structural and functional properties not present in any individual component thereby having a solid one-piece design capable of protecting interior components from tampering, blunt impact forces and water intrusion.

Viewing FIG. 1 of said invention MEDICAL CANNABIS AND CANNABINOIDS DELIVERY DEVICE's interior components of said Onboard Computer Processor 10, and said Evaluation Computer Processor 11, and said Power source 12 as well as external Digital Display 4, and Forward/Up Communication Button 5, and Fingertip Biometric Scanner 6, and Reverse/Down Communication Button 7; and Charging and Communication Port 8, and Heating Element Receptacle 9; and Vaporizer, Atomizer, Cartomizer, or a Clearomizer Chamber 2; and Nozzle 1.

Also viewing FIG. 1 reader can see Fingertip Biometric Scanner 6 that allows only the preprogrammed patient to access and operate MEDICAL CANNABIS AND CANNABINOIDS DELIVERY DEVICE. As said patient is prescribed an amount of medical cannabis or cannabinoids by said Medical Doctor or Pharmacist, they can communicates with said onboard computer processor 10 and input such as but not limited to dosage strength and time increments for usage as well as programming patients personal identification finger print into said onboard computer processer 10 by way of said Fingertip Biometric Scanner 6. My invention is now childproof and solves a prior art problem of unauthorized access and subsequent use.

Additionally, FIG. 1 shows said digital display 4 that is directly communicated to by the OCP 10 and alerts said patient when it's time to medicate based upon said prescribing doctor's prescription. Said digital display 4 also communicates with said patient by instructing them to inhale in the example of shallow breathing thus ensuring prescription tolerance are met.

As said patient inhales and depresses Fingertip Biometric Scanner 6 communicating with said onboard computer processer 10, said processor activates Heating Element Receptacle 9 which emits an electric charge creating a heat source for said Vaporizer, Atomizer, Cartomizer, or Clearomizer Chamber 2 which contains an amount of prescribed medicine. As said patient continues to draw through said nozzle 1 and inhaling said medical cannabis or cannabinoids, a portion of said vapors are directed through vapor sensing port 13, which communicates with said Evaluation Computer Processor (ECP) 11.

Said ECP 11 compares exiting vapor concentrate to said prescribed vapor concentrate located within said Evaluation Computer Processor (ECP) 11 memory and depending on the input recorded such as but not limited to a hard inhale, a soft inhale or a long, slow inhale or a hard, long inhale, said ECP 11 modulates an air, vapor, heat, and time duration program to deliver a reliably measurable dose of medical cannabis or cannabinoids equal to said patient prescription.

While the invention has been described with respect to certain specific embodiment, it will be appreciated that many modifications and changes may be made by those skilled in the art without departing from the spirit of the invention. It is intended, therefore, by the appended claims to cover all such modifications and changes as fall within the true spirit and scope of the invention. In particular, with respect to the above description, it is to be realized that the optimum dimensional relationships for the parts of the non-limiting exemplary embodiments may include variations in size, materials, shape, form, function and manner of operation.

1. A MEDICAL CANNABIS or CANNABINOIDS DELIVERY DEVICE (MCDD) comprising:
a tamper resistant water proof housing;
wherein said tamper resistant housing having a digital display; and
a Vaporizer, Atomizer, Cartomizer, or Clearomizer Chamber; and
a power source; and
an onboard computer processor, and
an evaluation computer processor; and
a fingertip biometric scanner; and
a heating element receptacle; and
one or more pass by vapor sensing ports.

2. The MEDICAL CANNABIS or CANNABINOIDS DELIVERY DEVICE (MCDD) of claim 1, further comprises:
a communication port to accept external or internal memory devices and communicate with such.

3. The MEDICAL CANNABIS or CANNABINOIDS DELIVERY DEVICE (MCDD) of claim 1, further comprising:
an access feature that is childproof and prevents unauthorized use and operation.

4. The MEDICAL CANNABIS or CANNABINOIDS DELIVERY DEVICE (MCDD) of claim 1, further comprising:
A means of interpreting the exhaust gas and comparing it to a prescription; and
A evaluation computer processor capable of communicating with a digital display; and
A onboard computer processor capable of communicating with a heating element; and
A digital display capable of communicating with user.

5. The MEDICAL CANNABIS or CANNABINOIDS DELIVERY DEVICE (MCDD) of wherein the medical cannabis or cannabinoids are Vaporizer, Atomizer, Cartomizer, or Clearomizer; and
Without regard to inhalation duration or strength producing a reliably measurable dose of medical cannabis or cannabinoids equal to said patient prescription.
